(19) United States
(12) Patent Application Publication
(10) Pub. No.: US 2025/0366871 A1
(43) Pub. Date: Dec. 4, 2025

(54) FEMORAL POSTERIOR REFERENCING SIZER

(71) Applicant: Zimmer, Inc., Warsaw, IN (US)

(72) Inventors: Kylie Hershberger, Warsaw, IN (US); Scott Dykema, Warsaw, IN (US); Brett Schultz, Warsaw, IN (US)

(21) Appl. No.: 18/869,065

(22) PCT Filed: Jun. 7, 2023
(86) PCT No.: PCT/US2023/024699

§ 371 Date: Nov. 25, 2024

Related U.S. Application Data
(60) Provisional application No. 63/351,619, filed on Jun. 13, 2022.

(51) Int. Cl. A61B 17/17; A61B 90/00; A61F 2/38; A61F 2/46

(52) U.S. Cl. CPC A61B 17/1764; A61F 2/4657; A61B 2090/067; A61F 2/3859; A61F 2002/4663; A61F 2002/4668

(57) ABSTRACT

An orthopedic instrument (10) for a knee arthroplasty, the instrument including: a base (12) having one or more paddles (36) configured to seat against and reference one or both of a medial condyle and a lateral condyle of a femur; a body (14) linked to the base for rotation relative thereto, wherein the body includes a recess and slot therein and a first indicia (24) along a first face of the body adjacent the slot; a post (20) received in the recess and moveable relative to the body, wherein the post has a second indicia (28) used with the first indicia to determine a posterior size of the femur; and an adjustment mechanism (18) having a third indicia (30) to indicate a rotation of the femur, wherein the adjustment mechanism is configured to rotate the body relative to the base to a desired orientation based upon the rotation of the femur.

### CLAIM OF PRIORITY

This application claims the benefit of U.S. Provisional Patent Application Ser. No. 63/351,619, filed on Jun. 13, 2022, the benefit of priority of which is claimed hereby, and which is incorporated by reference herein in its entirety.

### TECHNICAL FIELD

The present disclosure relates to knee arthroplasty. More particularly, the present disclosure relates to an instrument for use during a knee arthroplasty procedure.

### BACKGROUND

In a total knee arthroplasty (TKA) procedure, a patient's distal femur is resected and replaced with a prosthetic femoral implant, and the patient's proximal tibia is resected and replaced with a prosthetic tibial implant. The prosthetic femoral implant articulates with the prosthetic tibial implant to restore joint motion.

Many factors influence joint motion after the TKA procedure. The size and shape of each prosthetic implant will impact joint motion. To this end the femoral implant is carefully sized and the anterior-posterior dimension of the resected distal femur is determined using an anterior-posterior (AP) sizer. Additionally, the location and orientation of each prosthetic implant, which is determined by the location and orientation of the corresponding bone resections, will impact joint motion.

### OVERVIEW

The present patent application provides an exemplary instrument for TKA femoral anterior-posterior (AP) sizing. The instrument can also measure rotation of the femur to set rotation for the femoral implant so as to ensure alignment of the femoral implant with a patient's anatomy. The instrument can be used before resecting or otherwise manipulating the patient's knee joint to evaluate the size and alignment of the femur. The instrument can be used as an aid to plan the TKA procedure including for drilling to locate one or more resection guides used in resecting the femur.

In some TKA procedures, it can be complicated to size the femur and set femoral rotation. Most TKA procedures utilize different instruments for a right knee as opposed to a left knee. Furthermore, one instrument may be used for sizing the femur and another may be used to determine rotation of the femur. Thus, at least two sizers and sometime further instruments are included in an instrument system. Having additional instruments adds cost and complexity to the procedure.

The present inventors have recognized that the complexity of TKA procedures for sizing the femur, measuring and setting rotation can be reduced by providing a single instrument that can measure the anterior-posterior size of the femur and measure and set rotation. Furthermore, the instrument can be universal and can be used for both a right and a left knee. The present inventors also have recognized the instrument can be used to guide one or more drills for the drilling apertures in the femur. The instrument can eliminate the need for a dedicated guide such as a gap referencing drill guide. Thus, the instrument can provide for one or more drilled holes that can be used to mount cut guides, for example. In this manner, the number of surgical components can be reduced with the present instrument.

To further illustrate the knee arthroplasty instrument disclosed herein, a non-limiting list of examples is provided herein. The examples are termed aspects and techniques:

In some aspects, the techniques described herein relate to an orthopedic instrument for a knee arthroplasty, the instrument optionally including: a base having one or more paddles configured to seat against and reference one or both of a medial condyle and a lateral condyle of a femur; a body linked to the base for rotation relative thereto, wherein the body includes a recess and slot therein and a first indicia along a first face of the body adjacent the slot; a post received in the recess and moveable relative to the body, wherein the post has a second indicia used with the first indicia to determine a posterior size of the femur; and an adjustment mechanism having a third indicia to indicate a rotation of the femur, wherein the adjustment mechanism is configured to rotate the body relative to the base to a desired orientation based upon the rotation of the femur.

In some aspects, the techniques described herein relate to an instrument, further optionally including a plurality of retention mechanisms each including a ball and spring assembly, wherein the plurality of retention mechanisms are positioned between various components including at least the base and the body, the post and the body and the post and a stylus.

In some aspects, the techniques described herein relate to an instrument, wherein optionally the adjustment mechanism includes a dial with a handle configured for hand adjustment of the dial by a surgeon.

In some aspects, the techniques described herein relate to an instrument, wherein optionally the post includes an assembly including an outer sleeve, an inner shaft and a cap, wherein the outer sleeve translates but is rotationally fixed relative to the body but the inner shaft is configured for rotation relative to the outer sleeve and the body.

In some aspects, the techniques described herein relate to an instrument, further optionally including a stylus coupled to the post by the cap and moveable relative thereto, wherein the cap includes an aperture that receives a ball, and wherein the stylus includes a plurality of detents each configured to be engaged by the ball.

In some aspects, the techniques described herein relate to an instrument, wherein optionally the cap includes one or more grooves and one or more projections, and wherein the stylus includes one or more lateral projections each configured to be received in one of the one or more grooves.

In some aspects, the techniques described herein relate to an instrument, wherein optionally the inner shaft is configured to retain a spring that engages the ball, wherein the ball is biased by the spring against the stylus.

In some aspects, the techniques described herein relate to an instrument, wherein optionally the outer sleeve is rotationally fixed relative to the body by a projection that defines the second indicia, wherein the projection is captured in the slot.

In some aspects, the techniques described herein relate to an instrument, wherein optionally the body includes a plurality of guide apertures therein and the base includes a second plurality of guide apertures therein.

In some aspects, the techniques described herein relate to an instrument, wherein optionally the base includes a recess, a pawl and a slot, wherein the adjustment mechanism is a dial that is rotatably inserted in the recess and configured to selectively engage the pawl with a ratchet, and wherein the slot is configured to receive an arm that links the base to the body.

In some aspects, the techniques described herein relate to an instrument, wherein optionally the arm is engaged by the dial and can travel within an arcuate groove within the dial.

In some aspects, the techniques described herein relate to an instrument, wherein optionally the body via the adjustment mechanism is capable of eighteen degrees of rotation, inclusive, relative to the base.

In some aspects, the techniques described herein relate to an orthopedic instrument for a knee arthroplasty, the instrument optionally including: a base having one or more paddles configured to seat against and reference one or both of a medial condyle and a lateral condyle of a femur; a body linked to the base for rotation relative thereto, wherein the body includes a recess and slot therein and a first indicia along a first face of the body adjacent the slot; a post assembly received in the recess and moveable relative to the body, wherein the post assembly includes an outer sleeve, an inner shaft and a cap, wherein the outer sleeve translates but is rotationally fixed relative to the body but the inner shaft is configured for rotation relative to the outer sleeve and the body, and wherein the post has a second indicia used with the first indicia to determine a posterior size of the femur; and a stylus coupled to the post by the cap and moveable relative thereto.

In some aspects, the techniques described herein relate to an instrument, wherein optionally the cap includes an aperture that receives a ball, and wherein the stylus includes a plurality of detents each configured to be engaged by the ball.

In some aspects, the techniques described herein relate to an instrument, wherein optionally the cap includes one or more grooves and one or more projections, and wherein the stylus includes one or more lateral projections each configured to be received in one of the one or more grooves.

In some aspects, the techniques described herein relate to an instrument, wherein optionally the inner shaft is configured to retain a spring that engages the ball, wherein the ball is biased by the spring against the stylus.

In some aspects, the techniques described herein relate to an instrument, wherein optionally the outer sleeve is rotationally fixed relative to the body by a projection that defines the second indicia, wherein the projection is captured in the slot.

In some aspects, the techniques described herein relate to an instrument, further optionally including: an adjustment mechanism having a third indicia to indicate a rotation of the femur, wherein the adjustment mechanism is configured to rotate the body relative to the base to a desired orientation based upon the rotation of the femur; and a plurality of retention mechanisms each including a ball and spring assembly, wherein the plurality of retention mechanisms are positioned between various components including at least the base and the body and the post assembly and the body.

In some aspects, the techniques described herein relate to an orthopedic instrument for a knee arthroplasty, the instrument optionally including: a base having one or more paddles configured to seat against and reference one or both of a medial condyle and a lateral condyle of a femur; a body linked to the base for rotation relative thereto, wherein the body includes a recess and slot therein and a first indicia along a first face of the body adjacent the slot; a post received in the recess and moveable relative to the body, wherein the post has a second indicia used with the first indicia to determine a posterior size of the femur; a stylus coupled to the post and moveable relative thereto; an adjustment mechanism having a third indicia to indicate a rotation of the femur, wherein the adjustment mechanism is configured to rotate the body relative to the base to a desired orientation based upon the rotation of the femur; and a ball and spring assembly configured to retain the stylus with the post.

In some aspects, the techniques described herein relate to an instrument, wherein optionally the post includes cap having an aperture that receives the ball, wherein the stylus includes a plurality of detents each configured to be engaged by the ball, wherein the cap includes one or more grooves and one or more projections, and wherein the stylus includes one or more lateral projections each configured to be received in one of the one or more grooves.

The above aspects can be combined in any one or any combination of and/or can optionally be configured such that all elements or options recited are available to use or select from.

These and other examples and features of the present devices will be set forth in part in the following Detailed Description. This overview is intended to provide a summary of subject matter of the present patent application. It is not intended to provide an exclusive or exhaustive removal of the invention. The detailed description is included to provide further information about the present patent application.

### BRIEF DESCRIPTION OF THE DRAWINGS

In the drawings, like numerals can be used to describe similar elements throughout the several views. The drawings illustrate generally, by way of example, but not by way of limitation, various examples discussed in the present document.

FIG. 1 is a perspective view of a knee arthroplasty instrument in accordance with an example of the present disclosure.

FIG. 1A is an exploded view of the instrument of FIG. 1 in accordance with an example of the present disclosure.

FIG. 2A is a top plan view of a stylus of the instrument of FIG. 1, in accordance with an example of the present disclosure.

FIG. 2B is a side plan view of the stylus of FIG. 2A, in accordance with an example of the present disclosure.

FIG. 2C is a bottom plan view of the stylus of FIGS. 2A and 2B, in accordance with an example of the present disclosure.

FIG. 3 is a plan view of an anterior side of the instrument of FIG. 1, in accordance with an example of the present disclosure.

FIG. 3A is a perspective view of a posterior side of the instrument of FIG. 3, in accordance with an example of the present disclosure.

FIG. 3B is a plan view of a side of the instrument of FIG. 3, in accordance with an example of the present disclosure.

FIG. 3C is a first cross-sectional view of the instrument of FIG. 3, in accordance with an example of the present disclosure.

FIG. 3D is a second cross-sectional view of the instrument of FIG. 3, in accordance with an example of the present disclosure.

FIG. 3E is a third cross-sectional view of the instrument of FIG. 3, in accordance with an example of the present disclosure.

FIG. 4 is a plan view of an anterior side of a body of the instrument of FIG. 1, in accordance with an example of the present disclosure.

FIG. 4A is a cross-sectional view of the body of FIG. 4, in accordance with an example of the present disclosure.

FIG. 5 is a perspective view of a base of the instrument of FIG. 1, in accordance with an example of the present disclosure.

FIG. 5A is a plan view of an anterior side of the base of FIG. 5, in accordance with an example of the present disclosure.

FIG. 5B is a first cross-sectional view of the base of FIG. 5A, in accordance with an example of the present disclosure.

FIG. 5C is a second cross-sectional view of the base of FIG. 5, in accordance with an example of the present disclosure.

FIG. 5D is an enlarged view of a portion of the second cross-sectional view of FIG. 5C.

FIGS. 6A and 6B are plan views of sides of a linkage of the instrument of FIG. 1, in accordance with an example of the present disclosure.

FIG. 7A is a perspective view of a knob of the instrument of FIG. 1, in accordance with an example of the present disclosure.

FIG. 7B is a plan view of a posterior side of the knob of FIG. 7A, in accordance with an example of the present disclosure.

FIG. 8 is a perspective view of the instrument of FIG. 1 undergoing operation to size a femur, in accordance with an example of the present disclosure.

FIG. 9 is a perspective view of the instrument of FIG. 1 undergoing operation to set rotation for subsequent components by referencing the femur, in accordance with an example of the present disclosure.

FIG. 10 is a perspective view of the instrument of FIG. 1 undergoing operation to act as a drill guide with one or more apertures guiding drills in accordance with an example of the present disclosure.

### DETAILED DESCRIPTION

Example embodiments will now be described more fully with reference to the accompanying drawings. Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

As used herein, “proximal” refers to a direction generally toward the head of a patient, and “distal” refers to the opposite direction of proximal, i.e., away from the head of a patient. As used herein, the terms “anterior” and “posterior” should be given their generally understood anatomical interpretation. Thus, “posterior” refers to a rear of the patient, e.g., a back of the knee. Similarly, “anterior” refers to a front of the patient, e.g., a front of the knee. Thus, “posterior” refers to the opposite direction of “anterior.” Similarly, the terms “medial” and “lateral” should be given their generally understood anatomical interpretation. “Medial” refers to the inner part of the knee prosthesis (when in the implanted orientation) and “lateral” refers to the outer part. “Medial” refers to the opposite direction of “lateral.” “Varus” is defined as relating to, or being synonymous with “medial” or being relatively more medially disposed than a midline or other feature or component. “Valgus” is defined as relating to, or being synonymous with “lateral” or being relatively more laterally disposed than a midline or other feature or component.

As used herein the term “varus-valgus” means either varus-to-valgus or valgus-to-varus. Similarly, the terms “proximal-distal”, “medial-lateral” and “anterior-posterior” refer to either possible direction of reference for each term. Thus, for example, “proximal-distal” or “anterior/posterior” means either “proximal-to-distal” or “distal-to-proximal”.

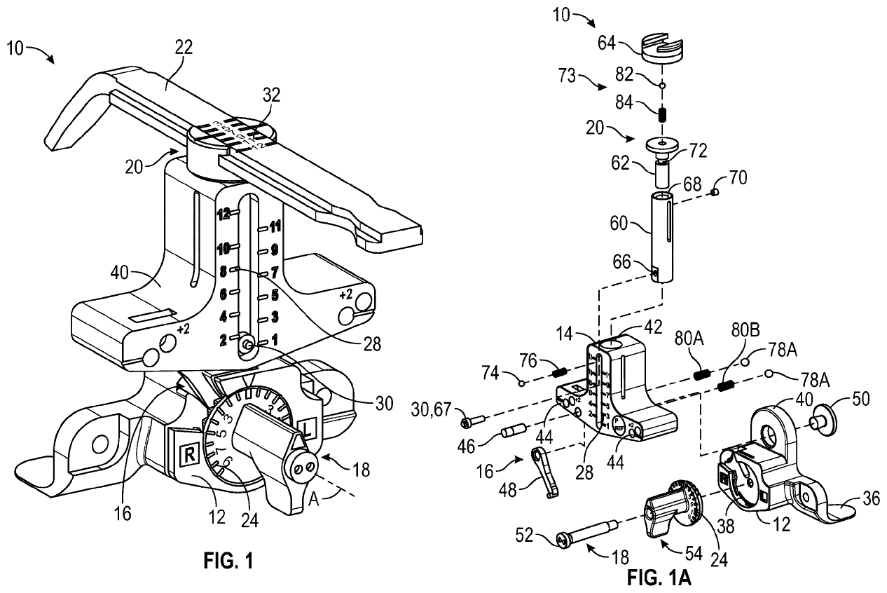

With reference to FIG. 1, an instrument 10 is shown. The instrument 10 can be configured for anterior-posterior (AP) sizing of the femur, can measure rotation of the femur and can set rotation for a femoral implant to ensure alignment of the femoral implant with a patient's anatomy.

The instrument 10 can include a base 12, a body 14, a linkage assembly 16, an adjustment mechanism 18, a post assembly 20 and a stylus 22.

The base 12 can be coupled to the body 14 via the linkage assembly 16. The body 14 can be moveable relative to the base 12 via the linkage assembly 16. The base 12 can be configured to receive the adjustment mechanism 18. The adjustment mechanism 18 can be coupled to the base 12 and can additionally be coupled to the linkage assembly 16. The adjustment mechanism 18 can be moveable relative to the base 12 and can drive movement of the linkage assembly 16 as further described herein.

The body 14 can be configured to receive the linkage assembly 16 and the post assembly 20. The post assembly 20 can be coupled with the stylus 22. The stylus 22 can be movable relative to the post assembly 20 and the body 14. The post assembly 20 can be moveable relative to the body 14 such as in a semi-constrained manner as further discussed herein.

The instrument 10 can be configured for use on either the left knee or the right knee of a patient. Furthermore, the instrument 10 is capable of measuring rotation of the knee (either left or right) up to 9 degrees of orientation as measured from a neutral position. This measured rotation (angle) can indicate a varus/valgus angle of the patient's knee joint and/or internal/external rotation of the patient's knee joint, for example.

The adjustment mechanism 18 can be adjusted through an 18 degree range of movement (9 degrees varus and 9 degrees valgus). The adjustment mechanism 18 can have first indicia 24 indicative of a rotation of the femur about axis A in FIG. 1. A second indicia 28 can be provided on the body 14 to measure and indicate an appropriate posterior size for a femoral implant (not shown). The second indicia 28 can be used in combination with a third indicia 30 on the post assembly 20. The stylus 22 can include a fourth indicia 32 to measure and indicate an appropriate an anterior size for the femoral implant (not shown). Using the second indicia 28, the third indicia 30 and the fourth indicia 32, the instrument 10 can measure the anterior to posterior distance referenced from posterior condyles (contacted by the base 12) of the femur (see subsequent figures) to an anterior cortex of the femur. The anterior cortex can be referenced with a tip of the stylus 22. This information, (the anterior-posterior distance) can be used to select a stock size of femoral implant. This stock size can be indicated with one or more of the indicia 28, 30, and/or 32 of the instrument 10.

FIG. 1A is an exploded view of the instrument 10 of FIG. 1 with the stylus 22 removed. Thus, FIG. 1A shows the base 12, the body 14, the linkage assembly 16, the adjustment mechanism 18 and the post assembly 20. The base 12 can include paddles 36 (only one is shown in FIG. 1A), a dial recess 38 and a flange 40. In addition to the second indicia 28, the body 14 can include a recess 42 and drill guide apertures 44. The linkage assembly 16 can include a fastener 46, an arm 48 and a second fastener 50. In addition to the first indicia 24, the adjustment mechanism 18 can include a fastener 52 and a dial 54. The post assembly 20 can include the third indicia 30, an outer sleeve 60, an inner shaft 62, and a cap 64. FIG. 1A shows further spring and ball assemblies of the instrument 10 that will be discussed in further detail subsequently.

The paddles 36 (only one shown in FIG. 1A) can comprise projections from a remainder of the base 12. The paddles 36 can be configured to abut or otherwise reference the lateral and medial posterior condyles of the femur. The dial recess 38 can be located in a first face of the base 12 on an opposing side of the base 12 from the paddles 36. The flange 40 can be on a femur facing side of the base 12 adjacent but proximal of the paddles 36.

The body 14 can have an inverted T-shape with wings extending laterally from a trunk or central portion. The recess 42 can be in the trunk and can be configured to receive at least a portion of the post assembly 20. The recess 42 can include one or more openings as further described herein including a slot along a first side of the body 14. The drill guide apertures 44 can be located in the wings of the body 14 and can extend through the body 14 from the first side to a femur facing side of the body 14.

The fastener 46 can couple the arm 48 to the body 14. However, the fastener 46 can allow for pivoting movement of the arm 48 relative to the body 14. The arm 48 can extend from the first end (coupled to the fastener 46) to a second end that interfaces with and is moveably engaged by the adjustment mechanism 18. In particular, the arm 48 can be configured as a follower of the adjustment mechanism 18 as further described and illustrated. The second fastener 50 can be received in an aperture in the flange 40 of the base 12 and can extend therethrough to be threaded or otherwise fastened to a femur facing side of the body 14. The fastener 50 can be configured to allow for pivoting movement of the body 14 relative to the base 12 as further illustrated and shown herein.

The fastener 52 of the adjustment mechanism 18 can hold the dial 54 rotatably in position relative to the base 12. As discussed previously, the dial 54 can be selectively rotatable up to 9 degrees (inclusive) clockwise and 9 degrees (inclusive) counterclockwise on the fastener 52 relative to the base 12. Thus, the dial 54 can be rotatable up to 18 degrees, inclusive on the fastener 52.

The outer sleeve 60 can have a first aperture 66 at or adjacent a distal end configured to receive a fastener 67 (e.g., a pin, screw or the like) that forms the third indicia 30. The outer sleeve 60 can include a second aperture 68 at or adjacent a proximal end configured to receive the inner shaft 62. The outer sleeve 60 can be linearly moveable proximal-distal relative to the body 14 but may not be rotatable relative to the body 14 as the fastener 67 is captured in the slot of the body 14. The inner shaft 62 can be rotatable within the outer sleeve 60. Thus, the inner shaft 62 can be rotatable relative to the outer sleeve 60 and the body 14. The inner shaft 62 can be maintained within the outer sleeve 60 by a fastener 70 that is received at least partially within a channel 72 or necked down region of the inner shaft 62 in addition to an aperture of the outer sleeve 60. The cap 64 can couple down onto and/or over a proximal end of the inner shaft 62. The cap 64 can be configured to receive the stylus 22 (not shown in FIG. 1A) and allow the stylus 22 to be moveable in a non-proximal direction (e.g., moveable anterior-posterior) thereto.

FIG. 1A shows spring and ball assemblies (also referred to herein as “retention mechanism(s)”) that can be utilized with the instrument 10. The retention mechanisms 73 can be used to reduce undesired slop or play between components. The retention mechanisms 73 can also in some cases facilitate a desired amount of engagement between components (e.g., can maintain a relative position of components when the components not being handled/manipulated by a surgeon). A first ball 74 and a first spring 76 can engage the outer sleeve 60 from a position mounted within the body 14. At least a second ball 78A, 78B and a second spring 80A and 80B can engage the body 14 from a position mounted within the base 12. A third ball 82 and a third spring 84 can be mounted in the inner shaft 62 and/or the cap 64 and the third ball 82 can pass through an aperture in the cap 64 to engage with the stylus 22 (not shown in FIG. 1A).

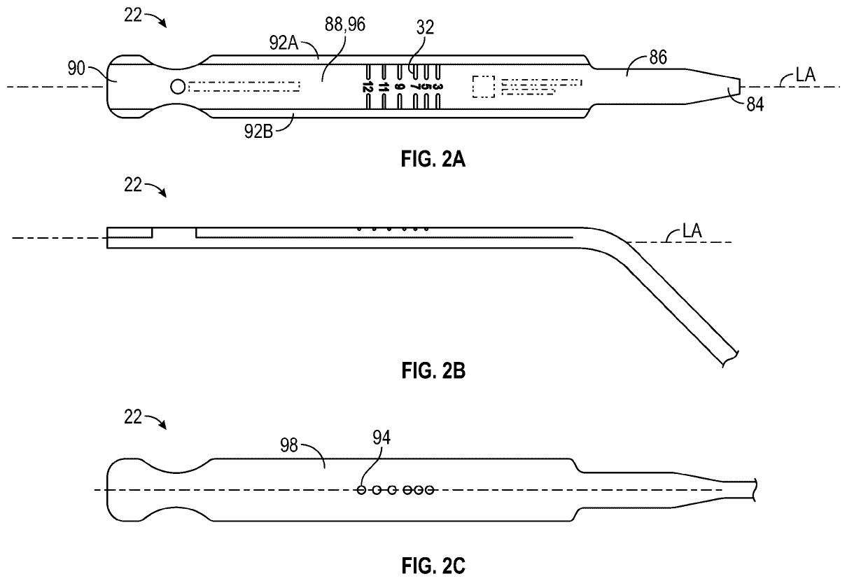

FIGS. 2A-2C show the stylus 22 in further detail. Referring to FIG. 2A, the stylus 22 can include a tip portion 84, a curved portion 86, a body 88 and an anterior end portion 90.

The body 88 can include the fourth indicia 32, lateral projections 92A and 92B and retention features 94 (FIG. 2C only).

The stylus 22 may or may not be used with the remainder of the instrument 10 (FIGS. 1 and 1A). The use of the stylus 22 can be at the discretion of the surgeon. The stylus 22 can have an elongate and relatively thin flat shape. The body 88 can extend along a longitudinal axis LA. The curved portion 86 can connect with the body 88 such as in an integral manner and can extend along the longitudinal axis but can also extend in a second direction such as distally to the tip portion 84. The tip portion 84 can connect with the curved portion 86. The tip portion 84 via the curved portion 86 and the body 88 can be configured to reference an anatomical point on the femur such as an anterior cortex of the femur.

The anterior end portion 90 can connect with the body 88 such as in an integral manner. The anterior end portion 90 can included a necked down or reduced material region to facilitate manipulation by a surgeon. The lateral projections 92A and 92B can extend to the anterior end portion 90. The lateral projections 92A and 92B can extend longitudinally along a length of the body 88 from the anterior end portion 90 to the curved portion 86. The lateral projections 92A and 92B form a reduced thickness region relative to a thickness of a remainder of the body 88. The lateral projections 92A and 92B are configured to interact with mating features such as grooves and/or projections on the proximal side of the cap 64 (FIGS. 1 and 1A). The features of the cap 64 in combination with the lateral projections 92A and 92B can retain the stylus 22 on the cap 64 but can allow for selective movement of the stylus 22 along the longitudinal axis LA. Thus, the stylus 22 may only be moveable along the longitudinal axis LA once the stylus 22 is coupled with the cap 64.

The fourth indicia 32 can be positioned on a first surface 96 (e.g., a top face) of the body 88. The fourth indicia 32 can reference a stock size of femoral implant based upon a distance the stylus 22 extends from the cap 64 (FIGS. 1 and 1A) to the anterior cortex or other feature on the femur. The fourth indicia 32 can include lines and/or numbers that can be aligned with a corresponding line (indicia) on the cap 64.

The stylus 22 can have a second surface 98. The second surface 98 can oppose the first surface 96. The retention features 94 can be formed in the body 88 and can extend into the second surface 98 having openings at the second surface 98. The retention features 94 can comprise detent or other features configured to receive a portion of the third ball 82 (FIG. 1A) extending from the cap 64. The retention features 94 can have positions along the longitudinal axis LA corresponding to the fourth indicia 32, for example. Thus, each of the retention features 94 can have a corresponding longitudinal position to one of the fourth indicia 32.

Retention force by the third spring 84 (FIG. 1A) via the third ball 82 can be exerted against the stylus 22. Simultaneous with the application of this force, the stylus 22 can be captured and retained by a generally opposing force of the grooves and projections with the lateral projections 92A and 92B.

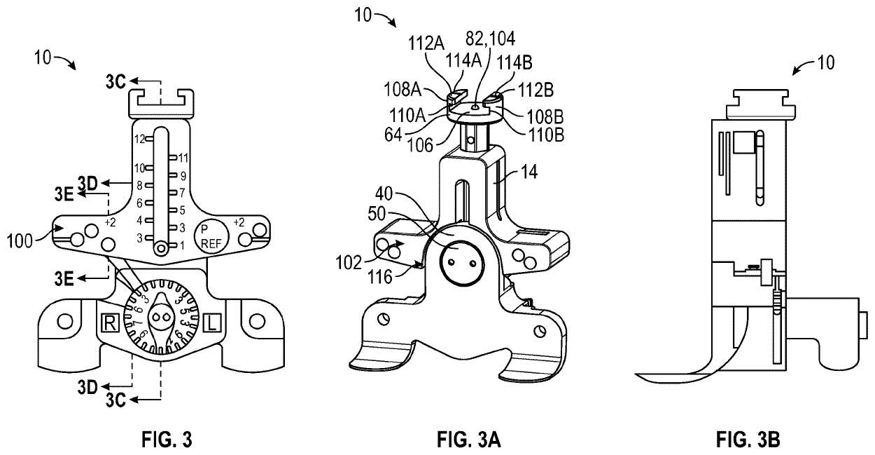

FIGS. 3-3E show various views of the instrument 10. FIGS. 3 shows a first side 100 (an anterior side when the instrument 10 is mounted to the femur). The components of the instrument 10 have been discussed previously in regards to FIGS. 1 and 1A.

FIG. 3A shows a second side 102 (a posterior side when the instrument 10 is mounted to the femur). FIG. 3A shows the construction of the cap 64 in further detail. In particular, the third ball 82 captured in a recess 104 in the cap 64. The recess 104 can extend into a first surface 106 in the cap 64. The third ball 82 can extend from the first surface 106 to engage the stylus 22 as previously discussed.

The cap 64 can include arms 108A and 108B. The arms 108A and 108B can extend from the first surface 106. The arms 108A and 108B can include grooves 110A and 110B, projections 112A and 112B and fifth indicia 114A and 114B.

The grooves 110A and 110B can be sized to receive the lateral projections 92A and 92B (FIGS. 2A-2C). The projections 112A and 112B can extend back toward one another forming a restriction or capture structure at a proximal side of the cap 64. The projections 112A and 112B can be engaged by portions of the stylus 22 (FIGS. 2A-2C) such as parts of the body 88 (FIGS. 2A-2C). The projections 112A and 112B can engaged by and capture the stylus 22 as previously discussed. This engagement can be facilitated by the third ball 82, which is forced against the stylus 22 as previously described. The fifth indicia 114A and 114B (e.g., lines) can be positioned on a proximal side of the projections 112A and 112B, respectively. The fifth indicia 114A and 114B can interact with the fourth indicia 32 of the stylus 22 as previously described.

FIG. 3A additionally shows a recess 116 in the body 14. The recess 116 is configured to receive the flange 40 therein. The recess 116 and flange 40 can be shaped to allow for pivoting movement of the body 14 relative to the base 12 on the second fastener 50.

FIG. 3B shows a side view of the instrument 10. FIG. 3C shows a cross-sectional view along line C-C of FIG. 3. FIG. 3C shows the base 12, the body 14, the linkage assembly 16, the adjustment mechanism 18 and the post assembly 20. As previously discussed the base 12 can include the paddles 36 (only one shown in FIG. 3C), the dial recess 38 and the flange 40. The body 14 can include the recess 42. The linkage assembly 16 can include the second fastener 50. The adjustment mechanism 18 can include the fastener 52 and the dial 54. The post assembly 20 can include the third indicia 32 (part of fastener 67 that can be simply a mark, line or the like), the outer sleeve 60, the inner shaft 62, and the cap 64. FIG. 3C also shows the first ball 74 and the first spring 76 and the third ball 82 and the third spring 84.

FIG. 3D shows a second cross-section along line D-D of FIG. 3. The view of FIG. 3D shows a portion of the body 14 and base 12 and additionally shows the second ball 78A and the second spring 80A. The second ball 78A can be forced against the base 12 (e.g., against the flange 40) by the second spring 80A. Engagement by the second ball 78A (and second ball 78B not shown) can reduce unwanted play or slop between the base 12 and the body 14.

FIG. 3E shows a third cross-section along line E-E of FIG. 3. FIG. 3E shows a portion of the body 14 along with the fastener 46 and part of the arm 48 of the linkage assembly 16.

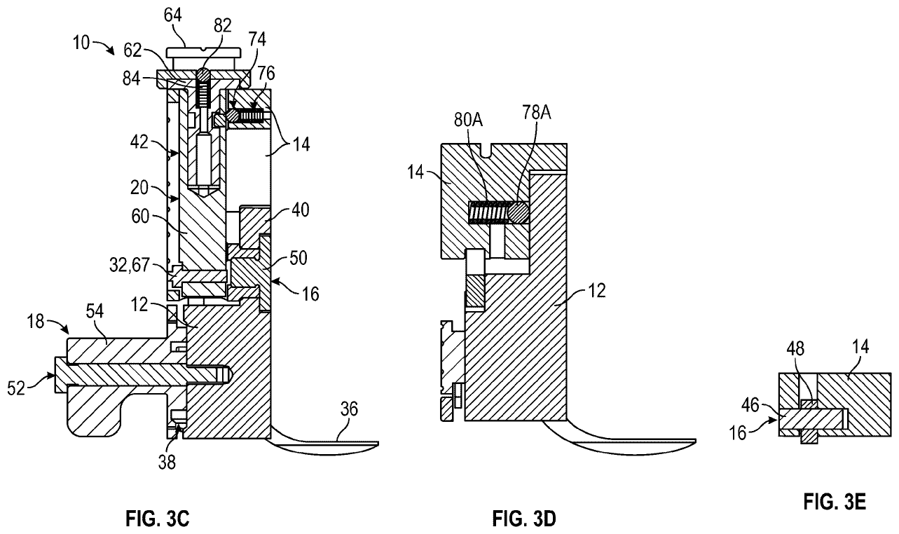

FIGS. 4 and 4A shows the body 14 and the post assembly 20 with the post assembly 20 shifted in position relative to FIG. 3C. Referring to FIG. 4, in addition to the to the second indicia 28, the recess 42 and the drill guide apertures 44, the body 14 can include wings 118A and 118B, second drill guide apertures 120A and 120B, a trunk 122 and a slot 124.

The wing 118A can define one of the drill guide apertures 44 and the second drill guide aperture 120A. The second drill guide aperture 120A can be offset a desired amount (e.g., 2 mm) from the drill guide aperture 44. The wing 118A can extend laterally from the trunk 122. Similarly, the wing 118B can define one of the drill guide apertures 44 and the second drill guide aperture 120B. The second drill guide aperture 120B can be offset a desired amount (e.g., 2 mm) from the respective drill guide aperture 44. The wing 118B can extend laterally from the trunk 122.

The trunk 122 can have an elongate shape proximal-distal and can have the second indicia 28 on a first face thereof. The trunk 122 can define the recess 42 therein. The slot 124 can extend from the first face to communicate with the recess 42.

As shown in FIGS. 4 and 4A, the third indicia 30 (part of the fastener 67) can be retained in the slot 124. The third indicia 30 can comprise a projection 126 (e.g., a head of the fastener 67 or other feature) that is captured by the slot 124. This arrangement of the captured projection 126 can restrain the outer sleeve 60 (FIG. 4A) from rotating but can allow for limited translation of the outer sleeve 60 (as dictated by the length of the slot 124) relative to the body 14.

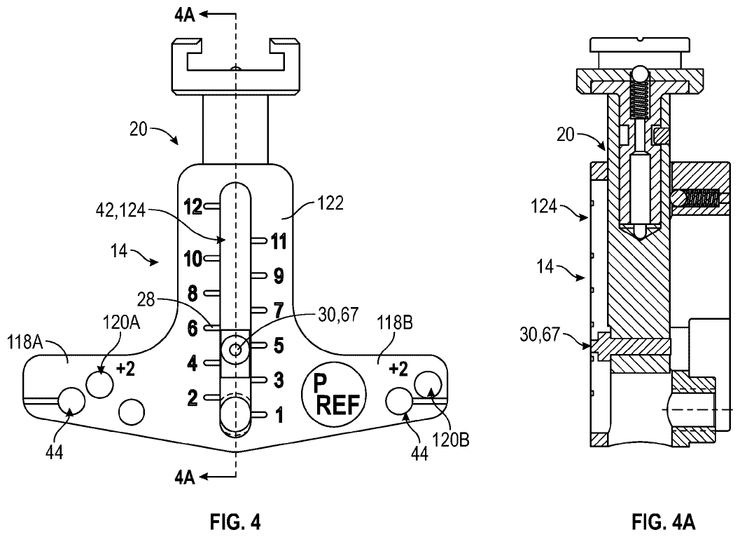

FIGS. 5-5D show further features of the base 12. These features can include the paddles 36 (only referenced in FIG. 5), the dial recess 38 and the flange 40 as previously discussed. Referring now to FIG. 5A, the base 12 can additionally include one or more drill guide apertures 128A and 128B, a slot 130 and a first aperture 132 and a second aperture 134.

A first of the drill guide apertures 128A can be located adjacent a first one of the paddles 36 and a second of the drill guide apertures 128B can be located adjacent a second one of the paddles 36. The drill guide apertures 128A and 128B are optional and may not be utilized to fixate the base 12 to the femur by some surgeons. The slot 130 can extend into the dial recess 38 from a proximal side thereof. The slot 130 can be configured to receive at least a portion of the arm 48 (FIG. 1A). The first aperture 132 can be located within the dial recess 38 such as at a center thereof. The first aperture 132 can be configured to thread or otherwise receive a distal portion of the fastener 52 (FIG. 1A). The second aperture 134 can be located in the flange 40 and can be configured to receive the second fastener 50 (FIG. 1A) therein.

FIG. 5B shows a cross-section along line B-B of FIG. 5A. FIG. 5C shows a cross-section through a portion of the dial recess 38. FIG. 5D is an enlarged view of a portion of the dial recess 38 of FIG. 5C.

FIG. 5B shows the dial recess 38, the flange 40, first aperture 132, and the second aperture 134. As shown in FIGS. 5B-5D, a projection or pawl 136 can be positioned within the dial recess 38 projecting from a side 138 of the base 12 that forms the dial recess 38. Although a single pawl 136 is shown in FIGS. 5B-5D, multiple pawls or other engagement features could be utilized in other examples. As best shown in FIG. 5D, the pawl 136 can be an arm have a tapered or angled ramp surface(s) 140 and a peak 142 adjacent the ramp surface(s) 140.

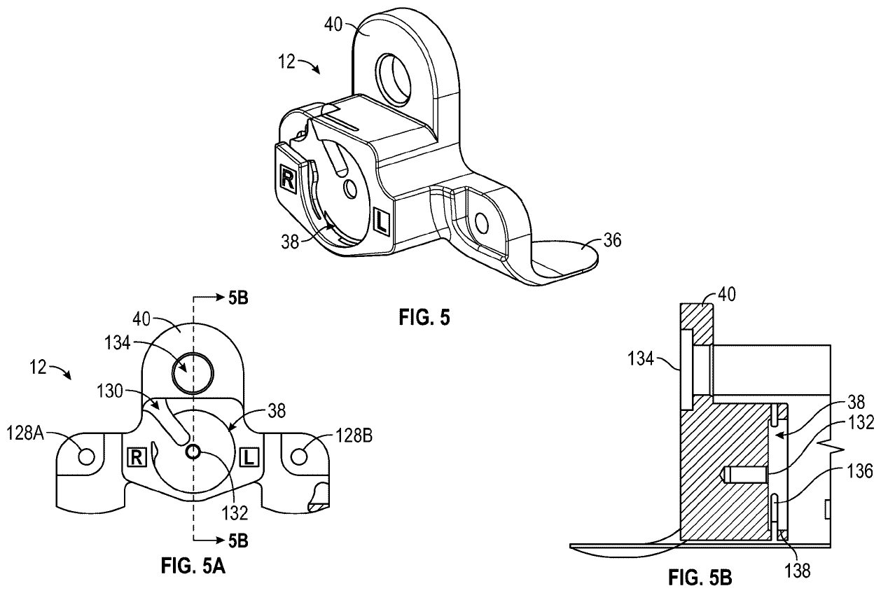

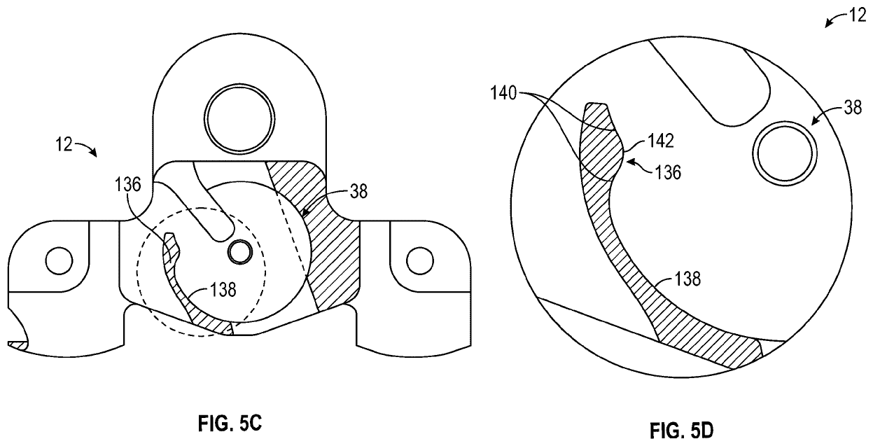

FIGS. 6A and 6B show aspects of the arm 48. As shown in FIG. 6A, the arm 48 can have an elongate length with a body 144, a first end 146 and a second end 148. The first end 146 can be opposed to second end 148. The first end 146 can include an aperture 150. The second end 148 can include a pin 152. The aperture 150 can be configured to receive the fastener 46 (FIG. 1A). The pin 152 can be configured to be received in a channel or groove 154 (FIG. 7A) within the dial 54. The groove 154 can have an arcuate or curved shape and can comprise a cam groove for example.

FIGS. 7A and 7B show the dial 54. As shown in FIG. 7A, the dial 54 includes the first indicia 24 along a face 155. The dial 54 can include a handle portion 156 (FIG. 7A) with an aperture 158 (FIG. 7A) configured to receive the fastener 52 (FIG. 1A) therein. The dial 54 can additionally include a ratchet 160 with detents 161 along a second side 162 thereof.

The detents 161 can be shaped to interact with the ramp surface(s) 140 and peak 142 (FIG. 5D) of the pawl 136 (FIG. 5D). The engagement of the pawl 136 with the detents 161 can retain the dial 54 relative to the base 12 (FIG. 1A) unless selectively rotated by the surgeon. The interaction and engagement of the arm 48 (FIGS. 6A and 6B), and in particular the pin 152 with the channel or groove 154 can cause pivoting rotation of the body 14 (FIG. 1A) with pivoting of the dial 54. The pin 152 and the channel or groove 154 can also limit rotation of the dial 54 when the pin 152 (FIGS. 6A and 6B) reaches one of the ends of the channel or groove 154. This limit to rotation of the dial 54 can be at substantially 9 degrees clockwise, inclusive, and 9 degrees counterclockwise, inclusive, rotation from a neutral position, for example.

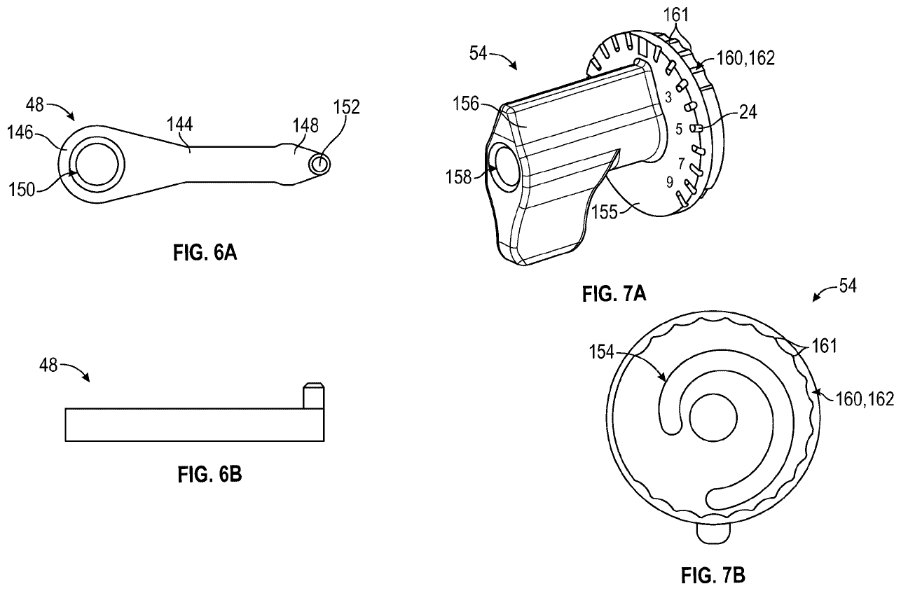

FIG. 8 shows the instrument 10 mounted to a distal portion 200 of a femur 202. The instrument 10 is in the process of measuring the proximal-distal size of the femur 202 using the post assembly 20 and stylus 22 as previously described. In brief, the base 12 can be positioned to contact the anterior and posterior condyles 206 (only one condyle shown in FIG. 8) via the paddles 36 (only one shown in FIG. 8) of the base 12. The post assembly 20 can be moved relative to body 14 to an appropriate posterior reference height. The stylus 22 can be moved relative to the body 14 and the post assembly 20 to reference a second part of the femur 202 such as by referencing an anatomic feature (e.g., the anterior cortex). The indicia discussed previously (the second, third, fourth, fifth, etc.) can used to measure the anterior-posterior size and select and appropriate stock size of femoral implant.

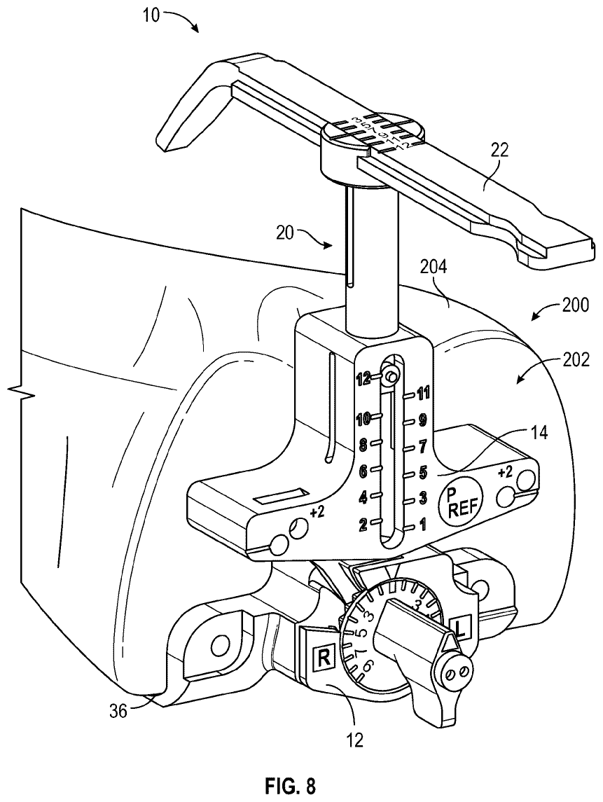

FIG. 9 shows the instrument 10 mounted to the distal portion 200 of the femur 202 and in the process of measuring a rotation of the femur 202. In particular, the adjustment mechanism 18 can be rotated clockwise relative to the base 12 to rotate the body 14 relative to the base 12 and the femur 202 via the linkage mechanism 16. The rotation of the femur 202 and the body 14 is indicated with the first indicia 24 on the dial 54. The rotation to 9 degrees shown in FIG. 9 is purely exemplary and is illustrated to show the rotational capability of the instrument 10 to measure rotation of the femur 202 for mounting of implant, guides, etc. Rotation of the femur is determined by visual alignment with Whiteside's Line which runs from the center of the intercondylar notch to the deepest point of the trochlear groove.

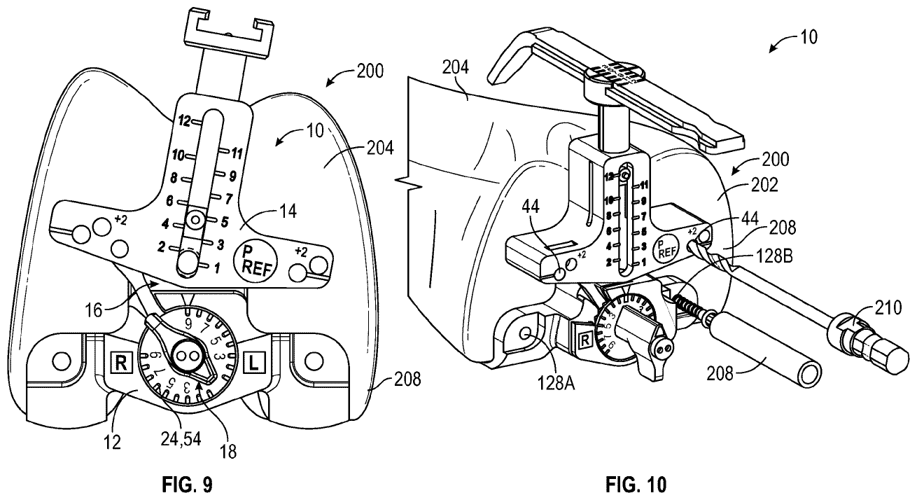

FIG. 10 shows the instrument 10 mounted to the distal portion 200 of the femur 202 and in the process of using the drill guide aperture 128B and the one of the drill guide apertures 44. Use of the drill guide aperture 128B and the one of the drill guide apertures 44 can occur once a proximal-distal size and the rotation of the femur 202 has been determined. In particular, the drill guide aperture 128B can be used to guide a first tool 208 (e.g., drill, pin, fastener, etc.) into the femur 202. The drill, pin, fastener, etc. once placed can be removed or can be used to aid in mounting the base 12 to the femur 202. A similar process can be repeated with the drill guide aperture 128A.

A second tool 210 (such a drill) can be guided by the one of the drill guide apertures 44 of the body 14 to create a recess in the femur 202. This recess can be used for mounting a cut guide (e.g., via a peg, pin, fastener or the like) or other instrument for performing resection or other surgical technique on the femur 202. The process of using the second tool 210 can be repeated using the second of the drill guide apertures 44 to guide drilling on a second lateral side of the body 14.

Optionally, the instrument 10 can include a set of modular accessories not specifically shown. Instrument 10 and the accessories can be provided together as a system. In this manner, a surgeon or another user can select a first accessory from the system and attach or use that first accessory with instrument 10. As the surgical procedure progresses, the surgeon can select a second accessory from the system and attach or utilize the second accessory with instrument 10 or with the first accessory. Thus, in some examples, the first accessory can be left in place when the second accessory is attached. A variety of different coupling mechanisms (e.g., dovetail joints) and locking mechanisms (e.g., keys, ball detents) can be used to selectively receive and retain the desired modular accessory on instrument 10. Additional information regarding modular accessories for instrument 10 can be found in U.S. Pat. No. 10,166,034B2 to Claypool et al., entitled “Knee Arthroplasty Instrument,” the disclosure of which is incorporated herein by reference in its entirety.

Components described herein unless otherwise indicated can be monolithic (i.e., single component structure) or can be coupled together in a known manner (e.g., with mechanical mechanisms as known in the art such as interference fit, coupling features such as threads, mating elements, etc.).

The above Detailed Description includes references to the accompanying drawings, which form a part of the detailed description. The drawings show, by way of illustration, specific examples in which the invention can be practiced. These examples are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.”

In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other examples can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed example. Thus, the following claims are hereby incorporated into the Detailed Description as examples or examples, with each claim standing on its own as a separate example, and it is contemplated that such examples can be combined with each other in various combinations or permutations. The scope of the invention should be determined with reference to the appended claims, along with the full scope of equivalents to which such claims are entitled.

1. An orthopedic instrument for a knee arthroplasty, the instrument comprising:
a base having one or more paddles configured to seat against and reference one or both of a medial condyle and a lateral condyle of a femur;
a body linked to the base for rotation relative thereto, wherein the body includes a recess and slot therein and a first indicia along a first face of the body adjacent the slot;
a post received in the recess and moveable relative to the body, wherein the post has a second indicia used with the first indicia to determine a posterior size of the femur; and
an adjustment mechanism having a third indicia to indicate a rotation of the femur, wherein the adjustment mechanism is configured to rotate the body relative to the base to a desired orientation based upon the rotation of the femur.

2. The instrument of claim 1, further comprising a plurality of retention mechanisms each comprising a ball and spring assembly, wherein the plurality of retention mechanisms are positioned between various components including at least the base and the body, the post and the body and the post and a stylus.

3. The instrument of claim 1, wherein the adjustment mechanism comprises a dial with a handle configured for hand adjustment of the dial by a surgeon.

4. The instrument of claim 1, wherein the post comprises an assembly including an outer sleeve, an inner shaft and a cap, wherein the outer sleeve translates but is rotationally fixed relative to the body but the inner shaft is configured for rotation relative to the outer sleeve and the body.

5. The instrument of claim 4, further comprising a stylus coupled to the post by the cap and moveable relative thereto, wherein the cap includes an aperture that receives a ball, and wherein the stylus includes a plurality of detents each configured to be engaged by the ball.

6. The instrument of claim 5, wherein the cap includes one or more grooves and one or more projections, and wherein the stylus includes one or more lateral projections each configured to be received in one of the one or more grooves.

7. The instrument of claim 6, wherein the inner shaft is configured to retain a spring that engages the ball, wherein the ball is biased by the spring against the stylus.

8. The instrument of claim 4, wherein the outer sleeve is rotationally fixed relative to the body by a projection that defines the second indicia, wherein the projection is captured in the slot.

9. The instrument of claim 1, wherein the body includes a plurality of guide apertures therein and the base includes a second plurality of guide apertures therein.

10. The instrument of claim 1, wherein the base includes a recess, a pawl and a slot, wherein the adjustment mechanism is a dial that is rotatably inserted in the recess and configured to selectively engage the pawl with a ratchet, and wherein the slot is configured to receive an arm that links the base to the body.

11. The instrument of claim 10, wherein the arm is engaged by the dial and can travel within an arcuate groove within the dial.

12. The instrument of claim 1, wherein the body via the adjustment mechanism is capable of eighteen degrees of rotation, inclusive, relative to the base.

13. An orthopedic instrument for a knee arthroplasty, the instrument comprising:
a base having one or more paddles configured to seat against and reference one or both of a medial condyle and a lateral condyle of a femur;
a body linked to the base for rotation relative thereto, wherein the body includes a recess and slot therein and a first indicia along a first face of the body adjacent the slot;
a post assembly received in the recess and moveable relative to the body, wherein the post assembly includes an outer sleeve, an inner shaft and a cap, wherein the outer sleeve translates but is rotationally fixed relative to the body but the inner shaft is configured for rotation relative to the outer sleeve and the body, and wherein the post has a second indicia used with the first indicia to determine a posterior size of the femur; and
a stylus coupled to the post by the cap and moveable relative thereto.

14. The instrument of claim 13, wherein the cap includes an aperture that receives a ball, and wherein the stylus includes a plurality of detents each configured to be engaged by the ball.

15. The instrument of claim 14, wherein the cap includes one or more grooves and one or more projections, and wherein the stylus includes one or more lateral projections each configured to be received in one of the one or more grooves.

16. The instrument of claim 15, wherein the inner shaft is configured to retain a spring that engages the ball, wherein the ball is biased by the spring against the stylus.

17. The instrument of claim 16, wherein the outer sleeve is rotationally fixed relative to the body by a projection that defines the second indicia, wherein the projection is captured in the slot.

18. The instrument of claim 17, further comprising:
an adjustment mechanism having a third indicia to indicate a rotation of the femur, wherein the adjustment mechanism is configured to rotate the body relative to the base to a desired orientation based upon the rotation of the femur; and
a plurality of retention mechanisms each comprising a ball and spring assembly, wherein the plurality of retention mechanisms are positioned between various components including at least the base and the body and the post assembly and the body.

19. An orthopedic instrument for a knee arthroplasty, the instrument comprising:
a base having one or more paddles configured to seat against and reference one or both of a medial condyle and a lateral condyle of a femur;
a body linked to the base for rotation relative thereto, wherein the body includes a recess and slot therein and a first indicia along a first face of the body adjacent the slot;
a post received in the recess and moveable relative to the body, wherein the post has a second indicia used with the first indicia to determine a posterior size of the femur;
a stylus coupled to the post and moveable relative thereto;
an adjustment mechanism having a third indicia to indicate a rotation of the femur, wherein the adjustment mechanism is configured to rotate the body relative to the base to a desired orientation based upon the rotation of the femur; and
a ball and spring assembly configured to retain the stylus with the post.

20. The instrument of claim 19, wherein the post includes cap having an aperture that receives the ball, wherein the stylus includes a plurality of detents each configured to be engaged by the ball, wherein the cap includes one or more grooves and one or more projections, and wherein the stylus includes one or more lateral projections each configured to be received in one of the one or more grooves.
